(19) United States
(12) Patent Application Publication
(10) Pub. No.: US 2013/0042482 A1
(43) Pub. Date: Feb. 21, 2013

(54) Skin Engaging Member Comprising An Anti-Irritation Agent

(71) Applicants: Valerie Jean Bradford, Framingham, MA (US); Michael Joseph Kwiecien, Scituate, MA (US); Timothy Woodrow Coffindaffer, Maineville, OH (US); Karen Marie Lammers, North Bend, OH (US); Edward Dewey Smith, III, Mason, OH (US); Jason Edward Cook, Anderson Township, OH (US); Brian Joseph Limberg, Milford, OH (US)

(72) Inventors: Valerie Jean Bradford, Framingham, MA (US); Michael Joseph Kwiecien, Scituate, MA (US); Timothy Woodrow Coffindaffer, Maineville, OH (US); Karen Marie Lammers, North Bend, OH (US); Edward Dewey Smith, III, Mason, OH (US); Jason Edward Cook, Anderson Township, OH (US); Brian Joseph Limberg, Milford, OH (US)

(21) Appl. No.: 13/586,427

(22) Filed: Aug. 15, 2012

Related U.S. Application Data
(60) Provisional application No. 61/524,095, filed on Aug. 16, 2011.

(51) Int. Cl. A61K 8/86; B29C 47/00; B26B 21/44; A61K 8/02; A61Q 19/00

(52) U.S. Cl. 30/41; 424/401; 514/188; 514/33; 514/18.8; 427/2.31

(57) ABSTRACT

A skin engaging member suitable for use in a hair removal device, said skin engaging member comprising an anti-irritation agent.

### CROSS REFERENCE TO RELATED APPLICATION

This application claims priority to U.S. Provisional Application No. 61/524,095 filed Aug. 16, 2011, the subject of which is hereby incorporated by reference in its entirety.

### BACKGROUND OF THE INVENTION

The use of shaving aids on razor blades to provide lubrication benefits during the shave is known. See e.g., U.S. Pat. Nos. 7,121,754; 6,298,558; 5,711,076; 5,134,775; 6,301,785; and U.S. Patent Publ. Nos. 2009/0223057, 2006/0225285. These shaving aids are also commonly referred to as lubrication strips. The delivery of additional benefits has also been attempted. For example, it has been described that cooling agents and/or essential oils can be included in the shaving aid to deliver a fresh and cool feel after contact. It has been reported, however, that a substantial amount of the essential oil can be lost due to volatilization prior to use. See U.S. Pat. No. 5,095,619. U.S. Pat. No. 5,713,131 attempts to fix this potential problem by introducing non-volatile cooling agents into the shave aid, such as non-volatile menthol analogs. Examples of other shave aids containing menthol and other actives are disclosed in U.S. Pat. Nos. 5,095,619; 6,298,558; 6,944,952; 6,295,733; 5,653,971 (disclosing a shaving aid which includes an improved shaving aid composite which contains an inclusion complex of a skin soothing agent, such as menthol, with a cyclodextrin); and 5,713,131 (disclosing Cooling Agent 10, WS-3, WS-23, Frescolat ML, Frescolat MGA and Menglytate). See also, U.S. Patent Pubs. 2007/0077331, 2008/031166, 2008/0300314A1; U.S. Pat. Nos. 5,451,404, and 7,482,373; and WO2007/036814A2.

Recently, it has been alleged that lubrication strips can include “a skin care ingredient selected from the group consisting of oils, vitamins, aloe, bisabalol, fruit extracts, green tea, antipruritic/counter irritant materials, antimicrobial/keratolytic materials, anti-fungal agents, anti-inflammatory agents, astringents, surfactants, rinsing aids, soaps, tackifiers, sunblocking agents, exfoliating agents, moisturizing agents, antiseptics, antioxidants, colorants, fragrances, and combinations of the foregoing.” See U.S. Patent Publ. No. 2006/0272155 at Abstract. Further, it has been reported that razors having a cleansing phase can include “an effective concentration of at least one compound selected from anti-acne actives, anti-wrinkle and anti-skin atrophy actives, skin barrier repair aids, cosmetic soothing aids, topical anesthetics, artificial tanning agents and accelerators, skin lightening actives, antimicrobial and antifungal actives, sunscreen actives, sebum stimulators, sebum inhibitors, antiperspirants, anti-glycation actives or mixtures thereof.” See U.S. Patent Publ. No. 2006/0225285 at paragraph 27.

Many different anti-irritation ingredients are known. There are so many ingredients capable of providing irritation impacting benefits in different media that a comprehensive list of ingredients would be very difficult to create. The challenge with incorporating these and other ingredients into shaving aids, particularly extruded and/or molded shaving aids, however, is that the processing conditions can negatively impact the effectiveness of these ingredients.

Many of the ingredients commonly included into skin care compositions, including many of those referenced by the above publications, are sensitive to elevated temperatures and pressures. This is particularly true of the ingredients which deliver a benefit to the skin, such as anti-irritation. This is due, in part, to the extrusion process which is commonly used to create conventional lubrication strips for use on many shaving razor products. Lube strip extrusion processing can include temperatures ranging from 160° C. to 180° C. Further, the feeds are often heated and mixed, then passed through one or more dies where a high amount of pressure can be applied. Many of the ingredients commonly described for skin care applications are often so volatile or sensitive that they can either vaporize or otherwise become denatured or otherwise damaged such that they no longer provide the desired benefit altogether, or become considerably less effective or potent. As such, there remains a need resilient anti-irritation agent which can be used in a conventionally extruded shaving aid yet still deliver a meaningful desired benefit to skin.

### SUMMARY OF THE INVENTION

One aspect of this invention relates to a skin engaging member, suitable for use with a hair removal device, such as a razor or depilatory and scraping tool, said skin engaging member comprising a matrix comprises at least one of: a water soluble polymer, an emollient, and a mixture thereof; at least one anti-irritation agent.

Another aspect of the invention relates to a method of making a skin engaging member comprising the steps of: providing a polymeric matrix with at least one anti-irritation agent.

### BRIEF DESCRIPTION OF THE DRAWINGS

FIG. 1 is a perspective view of a razor cartridge which includes a skin engaging member of the present invention. FIG. 2 is a sectional view taken along line 2-2 of FIG. 1. FIG. 3 is a side elevation view of second type of skin engaging member of the present invention.

### DETAILED DESCRIPTION OF THE INVENTION

### I. Anti-Irritation Agent

In one embodiment the skin engaging member comprising a shaving aid composition of the present invention further comprises an anti-irritation agent. The anti-irritation agent can be pyrithione or a polyvalent metal salt of pyrithione, or a mixture thereof. Any form of polyvalent metal pyrithione salts may be used, including platelet and needle structures. Preferred salts for use herein include those formed from the polyvalent metals magnesium, barium, bismuth, strontium, copper, zinc, cadmium, zirconium and mixtures thereof, more preferably zinc. Even more preferred for use herein is the zinc salt of 1-hydroxy-2-pyridinethione (known as “zinc pyrithione” or “ZPT”); more preferably ZPT in platelet particle form, wherein the particles have an average size of up to about 20 μm, preferably up to about 5 μm, more preferably up to about 2.5 μm.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

Preferred embodiments include from 0.01% to 5% of an anti-irritation agent; alternatively from 0.05% to 2%, alternatively from 0.1% to 1%, alternatively from 0.2% to about 0.7%, alternatively about 0.5%.

In one embodiment, the anti-irritation agent is a solid particle in the form of a platelet as described here.

Those of skill in the art will understand that the anti-irritation agent of the present invention can also have other benefits which may be desirable from a skin care composition, including but not limited to malodor control and/or anti-bacterial benefits depending on whether the composition is left on skin or rinsed off.

The composition of the present invention optionally includes an effective amount of a zinc salt. Preferred embodiments of the present invention include an effective amount of a zinc salt having an aqueous solubility within the composition of less than about 25%, by weight, at 25° C., more preferably less than about 20%, more preferably less than about 15%. Preferred embodiments of the present invention include from 0.001% to 10% of a zinc salt, more preferably from 0.01% to 5%, more preferably still from 0.1% to 3%. In a preferred embodiment, the zinc salt has an average particle size of from 100 nm to 30 μm.

Examples of zinc salts useful in certain embodiments of the present invention include the following: Zinc aluminate, Zinc carbonate, Zinc oxide and materials containing zinc oxide (i.e., calamine), Zinc phosphates (i.e., orthophosphate and pyrophosphate), Zinc selenide, Zinc sulfide, Zinc silicates (i.e., ortho- and meta-zinc silicates), Zinc silicofluoride, Zinc Borate, Zinc hydroxide and hydroxy sulfate, zinc-containing layered materials and combinations thereof.

In embodiments having an anti-irritation agent and a zinc salt, the ratio of zinc salt to anti-irritation agent is preferably from 5:100 to 5:1; more preferably from about 2:10 to 3:1; more preferably still from 1:2 to 2:1.

Without intending to be bound by theory, it is believed that these anti-irritation agents can provide various benefits including reduction or control of irritation as well as certain malodor control. In one embodiment, the composition further comprises other agents such as malodor control agents. The malodor active of the present invention is capable of providing an antimicrobial benefit. Such malodor actives are capable of destroying microbes, preventing the development of microbes or preventing the pathogenic action of microbes. A safe and effective amount of a malodor active may be added to the intimate cleansing product, at from about 0.001% to about 10%, or from about 0.01% to about 5%, or from about 0.05% to about 2%, or from about 0.1% to about 1%, or from about 0.3% to about 0.7%, or about 0.5% by weight of the composition.

Examples of malodor actives include β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, clotrimazole, and mixtures thereof.

### II. Thermally Resilient Skin Care Active

In another embodiment, the skin engaging member further comprises at least one thermally resilient skin care active. Thermally resilient skin care actives, as defined herein, are compositions which are suitable for dermatological use and are capable of surviving conventional shaving aid (skin engaging member) extrusion conditions while remain sufficiently active to provide their desired benefit on skin. In one embodiment, the thermally resilient skin care active substantially survives extended amounts of time at elevated temperatures such as above about 120° C. or even as high as about 200° C. Substantially survives, as defined herein, means that at least 25% by weight of the material retains its molecular structure after processing into the final product, which can be determined by an appropriate analytical method such as LC-MS or GC-MS, preferably at least 50%, preferably at least 75%, preferably at least 90%, after it is subjected to the previously mentioned elevated temperatures. Details on how to measure molecular survival are provided in section I(f), below.

In one embodiment, the thermally resilient skin care active has phase transition temperature into a gas (i.e. boiling point or sublimation point) which is greater than the extrusion temperature of the shaving aid forming the skin engaging member. In another embodiment, the phase transition temperature into a gas is at least 120° C., or at least about 150° C., or at least about 180° C., up to about 200° C.

Those of skill in the art will understand that skin engaging members include shaving aids which are also commonly referred to as lubricating strips. These skin engaging members can be used on hair removal devices such as shaving razors.

In one embodiment, the at least one thermally resilient skin care active comprising at least one of: a glycyrrhizinate salt; a surfactant derived from a triglyceride; a ceramide, esters of pyrrolidone carboxylic acid; a peptide; or a mixture thereof. In one embodiment, the level of the at least one thermally resilient skin care active can be from about 0.1% to about 20%, or from about 1% to about 15%, or from about 5% to about 10%, by weight of the skin engaging member.

a. Glycyrrhizinate Salt

Glycyrrhizinate salt is widely used anti-inflammatory agent isolated from the licorice root. It is metabolized to glycyrrhetic acid, which inhibits 11 beta-hydroxysteroid dehydrogenase and other enzymes involved in the metabolism of corticosteroids. In one embodiment, the glycyrrhizinate salt is a dipotassium salt, as shown below:

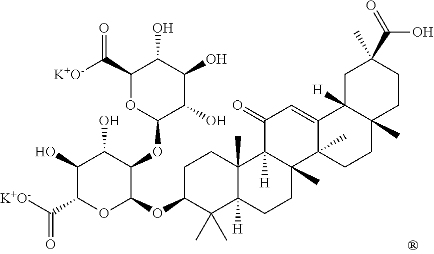

It may be purchased from: Mafco, Sandream or Barnet. Other salts of glycyrrhizinate may also be used, including but not limited to ammonium, sodium, and alkanolamine (e.g., triethanolamine) salts. One example of a glycyrrhizinate salt is Net-DG® by Barnet.

b. Surfactant Derived from a Triglyceride

The multi-active of the present invention also comprises a surfactant derived from a triglyceride. The triglyceride of the present invention comprises a multiple fatty acids: Oleic fatty acid, linoleic fatty acid, linoleic acid and combinations thereof. Preferably the majority of the fatty acids are the oleic and linoleic fatty acids. The anionic surfactant is formed by isolating the alkyl chains from the triglyceride into fatty acids, methyl ethers, or fatty alcohols by hydrolysis. The isolated alkyl chains are then ethoxylated. The ethoxylated alkyl chain is then carboxylated. In one embodiment, the anionic surfactant comprises the product of ethoxylating then carboxylating oleic acid. In one embodiment the anionic surfactant is Sodium Polyethylene glycol 7 Olive Oil Carboxylate. In one embodiment, the triglyceride is predominantly unsaturated. “Predominantly unsaturated” as defined herein means that at least 50% by weight of the triglyceride used to make the surfactant is not saturated. Those of skill in the art will also understand that predominantly unsaturated triglycerides include monounsaturated fats and polyunsaturated fats, and tend to be liquid at room temperature. One suitable surfactant derived from a triglyceride is Olivem® 450 and 460 by B&T Company of Milan, Italy.

Suitable predominantly unsaturated triglycerides include plant derived oils such as castor oil, corn oil, oiticica oil, olive oil, lin seed oil, cotton seed oil, palm oil, peanut oil, grape seed oil (canola), rice bran oil, safflower oil, sesame oil, sorghum oil, tall oil, tongue oil, soy bean oil, sunflower oil, almond oil, other similarly related vegetable oils and mixtures thereof. Notably, these triglycerides are considered to be saponifiable, thereby resulting in the anionic surfactant. In one preferably embodiment, the triglyceride is olive oil, preferably a sodium polyethylene glycol 7 olive oil carboxylate.

c. Ceramides

Ceramides are often considered a class of skin lipids. Skin lipids perform an integral function of maintaining the water holding ability of the stratum corneum, regulating skin health. Ceramides, the primary component of skin lipids, and are comprised of sphingolipids consisting of a long chain amino alcohol (sphingosine or one of it's derivatives) to which a long chain fatty acid is linked via an amide bond. Natural or synthetic sources can be used, as can pseudo-ceramides. Preferably, the skin care active herein comprises ceramide, cholesterol, sphingosine, or mixtures thereof. Non-limiting examples of commercially available blends of skin lipids suitable for use herein include SK Influx from Cosmoferm.

Ceramides are also described in Arch. Dermatol, Vol 123, 1381-1384, 1987, or those described in French Patent FR-2,673,179; fatty acid polyesters such as, sucrose 65 pentalaurate, sucrose tetraoleate, sucrose pentaerucate, sucrose tetraerucate, sucrose pentatallowate, sucrose triapeate, sucrose tetrapeate, sucrose pentarapeate, sucrose tristearate, and sucrose pentastearate, and mixtures thereof; polypeptides and amino acids consisting of basic amino acids, particularly arginine.

d. Esters of Pyrrolidone Carboxylic Acid (PCA)

Pyrrolidone carboxylic acid are also referred to as PCA or pyroglutamic acid. Esters of pyroglutamic acid, as described by Lever Brothers Company in U.S. Pat. No. 4,774,255, such as: pyroglutamic acid n-hexyl ester, pyroglutamic acid n-octyl ester, ethyl-2-[pyroglutamoyloxy]-n-propionate, linoleyl-2-[pyroglutamoyloxy]-n-caprylate,lauryl-2-[pyroglutamoyloxy]-n-caprylate, stearyl-2[pyrogluta-moyloxy]-n-caprylate, glyceryl mono(2[pyroglutamoyloxy]-n-propionate), glyceryl mono(2[pyroglutamoyloxy]-n-caprylate), and glyceryl di(2[pyroglutamoyloxy]-n-propionate); (viii) Aryl-substituted ethylenes, as described by Unilever in EP-A-0 403 238, (ix) Mono N-acylated amino acids, as described by Unilever in EP-A-0 415 598, especially: N-acetyl glycine (x) Saturated or unsaturated aliphatic alcohols having an odd number of carbon atoms of from 3 to 25 in number, especially: n-nonyl alcohol, (xi) Saturated or unsaturated aliphatic carboxylic acids having an odd number of carbon atoms of from 3 to 25 in number, especially: nonanoic acid; and (xii) mixtures thereof. In one embodiment, the ester of pyrrrolidone carboxylic acid is octyldodecyl ester of PCA, commercially available as CERAMIDONE® from Solabia.

e. Peptides

The compositions of the present invention may contain a safe and effective amount of a peptide, including but not limited to, di-, tri-, tetra-, and penta-peptides and derivatives.

As used herein, “peptide” refers to peptides containing ten or fewer amino acids and their derivatives, isomers, and complexes with other species such as metal ions (e.g., copper, zinc, manganese, magnesium, and the like). As used herein, peptide refers to both naturally occurring and synthesized peptides. Also useful herein are naturally occurring and commercially available compositions that contain peptides. Preferred peptides contain at least one basic amino acid (e.g., histidine, lysine, arginine). More preferred peptides are the dipeptide carnosine (beta-ala-his), the tripeptide gly-his-lys, the tripeptide his-gly-gly, the tripeptide gly-gly-his, the tripeptide gly-his-gly, the tetrapeptide gly-gln-pro-arg, the pentapeptide lys-thr-thr-lys-ser, and metal complexes of the above, e.g., copper complex of the tripeptide his-gly-gly (also known as Iamin). Other suitable peptides include Peptide CK (arg-lys-arg); Peptide CK+(ac-arg-lys-arg-NH2); and Peptide E, arg-ser-arg-lys. A preferred commercially available tripeptide derivative-containing composition is Paltenex® or Maxi-Lip®, which contains 1000 ppm of palmitoyl-gly-his-lys and is commercially available from Sederma, France. A preferred commercially available pentapeptide derivative-containing composition is Matrixyl®, which contains 100 ppm of palmitoyl-lys-thr-thr-lys-ser and is commercially available from Sederma, France. A preferred commercially available tetrapeptide derivative-containing composition is Rigin®, which contains 500 ppm of palmitoyl-gly-gln-pro-arg and is commercially available from Sederma, France.

Peptide derivatives useful herein include lipophilic derivatives, preferably palmitoyl derivatives. Preferably, the peptide is selected from palmitoyl-lys-thr-thr-lys-ser, palmitoyl-gly-his-lys, palmitoyl-gly-gln-pro-arg, their derivatives, and combinations thereof. Another example of suitable peptides include Pal-GHK from Genscript.

### III. Additional Skin Care Active Ingredients

Various skin care actives (“actives”) which are commonly used for topical application can also used in addition to the at least one thermally resilient skin care active. The at least one thermally resilient skin care active and the additional actives can each be included in the skin engaging member as a neat product and/or in an encapsulate.

The active ingredient can also be one or more skin care actives suitable for topical use. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, camphor, eucalyptus oil, eugenol, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, fatty alcohols and fatty acids, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, skin-conditioning agents, skin soothing and/or healing agents and derivatives, skin treating agents, thickeners, and vitamins and derivatives thereof. Additional non-limiting examples of additional suitable skin treatment actives are included in U.S. Patent Publ. No. 2003/0082219 in Section I (i.e. hexamidine, zinc oxide, and niacinamide); U.S. Pat. No. 5,665,339 at Section D (i.e. coolants, skin conditioning agents, sunscreens and pigments, and medicaments); and U.S. Patent Publ. No. 2005/0019356 (i.e. desquamation actives, anti-acne actives, chelators, flavonoids, and antimicrobial and antifungal actives). It should be noted, however, that many materials may provide more than one benefit, or operate via more than one mode of action. Therefore, classifications herein are made for the sake of convenience and are not intended to limit the active to that particular application or applications listed.

f. Cooling Agents

Non-limiting examples of suitable cooling agents include: L-menthol; p-methane-3,8-diol; Isopulegol; Menthoxypropane-1,2,-diol; Curcumin; Menthyl Lactate (such as Frescolat ML by Symrise); Gingerol; Icilin; Tea Tree Oil; Methyl Salicylate; Camphor; Peppermint Oil; N-Ethyl-p-menthane-3-carboxamide; Ethyl 3-(p-menthane-3-carboxamido)acetate; 2-Isopropyl-N,2,3-trimethylbutyramide; Menthone glycerol ketal, Menthone Glyerine Acetal; Coolact 10; and mixtures thereof. These and other cooling agents are known and described in various publications, such as U.S. Patent Publ. No. 2008/0300314A1, U.S. Pat. Nos. 5,451,404 and 7,482,373. In yet another embodiment, the cooling agent comprises one or more of the cooling agents previously described for use in various shave aids. See e.g., U.S. Pat. Nos. 5,095,619; 5,713,131; 5,095,619; 5,653,971; 6,298,558; 6,944,952; and 6,295,733.

In one embodiment, the skin engaging member further comprises one or more cooling agents. It is now well established that sensations such as cool or cold can be attributed to activation of receptors at peripheral nerve fibers by a stimulus such as low temperature or a chemical coolant, which produces electrochemical signals that travel to the brain, which then interprets, organizes and integrates the incoming signal(s) into a perception or sensation. Different classes of receptors have been implicated in sensing cold temperatures or chemical coolant stimuli at mammalian sensory nerve fibers. Among these receptors, a major candidate involved in sensing cold has been identified and designated as cold- and menthol-sensitive receptor (CMR1) or TRPM8. The TRPM8 nomenclature for the receptor comes from its characterization as a non-selective cation channel of the transient receptor potential (TRP) family that is activated by stimuli including low temperatures, menthol and other chemical coolants. However, the precise mechanisms underlying the perception of a pleasant cooling sensation on skin or oral surfaces are presently not clearly understood. While it has been demonstrated that the TRPM8 receptor is activated by menthol and other coolants, it is not fully understood what other receptors may be involved and to what extent these receptors need to be stimulated or perhaps suppressed in order that the overall perceived sensation would be pleasant, cooling and refreshing. For example, menthol is widely used as a cooling agent, but menthol can also produce other sensations including tingling, burning, prickling and stinging as well as a minty smell and bitter taste. Thus, it can be inferred that menthol acts on many different receptors, including cold, warm, pain and taste receptors. However, it is not readily discernible how to isolate which receptor activities would result in a specific sensation such as pleasant cooling without the undesirable sensations such as bitterness or irritation. Neither is it apparent how to control the activity of coolants or other sensory agents such that only the desired sensation is elicited from use of a particular sensory agent. As such, the present invention is focused on the addition of specific synthetic derivatives of cyclohexane (described above) to act as sensates to deliver cooling benefit to users during the hair removal process. Additional sensates can be used to further supplement the cooling feel.

A large number of coolant compounds of natural or synthetic origin are known. The most well-known compound is menthol, particularly l-menthol, which is found naturally in peppermint oil, notably of Mentha arvensis L and Mentha viridis L. Of the isomers of menthol, the 1-isomer occurs most widely in nature and is typically what is referred by the name menthol having coolant properties. L-menthol has the characteristic peppermint odor, has a clean fresh taste and exerts a cooling sensation when applied to the skin and mucosal surfaces. Other isomers of menthol (neomenthol, isomenthol and neoisomenthol) have somewhat similar, but not identical odor and taste, i.e., some having disagreeable notes described as earthy, camphor, musty. The biggest difference among the isomers is in their cooling potency. L-menthol provides the most potent cooling, i.e., having the lowest cooling threshold of about 800 ppb, i.e., the concentration where the cooling effect could be clearly recognized. At this level, there is no cooling effect for the other isomers. For example, d-neomenthol is reported to have a cooling threshold of about 25,000 ppb and 1-neomenthol about 3,000 ppb. [R. Emberger and R. Hopp, “Synthesis and Sensory Characterization of Menthol Enantiomers and Their Derivatives for the Use in Nature Identical Peppermint Oils,” Specialty Chemicals (1987), 7(3), 193-201]. This study demonstrated the outstanding sensory properties of l-menthol in terms or cooling and freshness and the influence of stereochemistry on the activity of these molecules.

Among synthetic coolants, many are derivatives of or are structurally related to menthol, i.e., containing the cyclohexane moiety, and derivatized with functional groups including carboxamide, ketal, ester, ether and alcohol. Examples include the ρ-menthanecarboxamide compounds such as N-ethyl-ρ-menthan-3-carboxamide, known commercially as “WS-3”, and others in the series such as WS-5 (N-ethoxycarbonylmethyl-ρ-menthan-3-carboxamide), and WS-14 (N-tert-butyl-ρ-menthan-3-carboxamide). Examples of menthane carboxy esters include WS-4 and WS-30. An example of a synthetic carboxamide coolant that is structurally unrelated to menthol is N,2,3-trimethyl-2-isopropylbutanamide, known as “WS-23”. Additional examples of synthetic coolants include alcohol derivatives such as 3-(1-menthoxy)-propane-1,2-diol known as TK-10, isopulegol (under the tradename Coolact P) and ρ-menthane-3,8-diol (under the tradename Coolact 38D) all available from Takasago; menthone glycerol acetal known as MGA; menthyl esters such as menthyl acetate, menthyl acetoacetate, menthyl lactate known as Frescolat® supplied by Haarmann and Reimer, and monomenthyl succinate under the tradename Physcool from V. Mane. TK-10 is described in U.S. Pat. No. 4,459,425 to Amano et al. Other alcohol and ether derivatives of menthol are described e.g., in GB 1,315,626 and in U.S. Pat. Nos. 4,029,759; 5,608,119; and 6,956,139. WS-3 and other carboxamide cooling agents are described for example in U.S. Pat. Nos. 4,136,163; 4,150,052; 4,153,679; 4,157,384; 4,178,459 and 4,230,688. Additional N-substituted ρ-menthane carboxamides are described in WO 2005/049553A1 including N-(4-cyanomethylphenyl)-ρ-menthanecarboxamide, N-(4-sulfamoylphenyl)-ρ-menthanecarboxamide, N-(4-cyanophenyl)-ρ-menthanecarboxamide, N-(4-acetylphenyl)-ρ-menthanecarboxamide, N-(4-hydroxymethylphenyl)-ρ-menthanecarboxamide and N-(3-hydroxy-4-methoxyphenyl)-ρ-menthanecarboxamide. Other N-substituted ρ-menthane carboxamides include amino acid derivatives such as those disclosed in WO 2006/103401 and in U.S. Pat. Nos. 4,136,163; 4,178,459 and 7,189,760 such as N-((5-methyl-2-(1-methylethyl)cyclohexyl)carbonyl)glycine ethyl ester and N-((5-methyl-2-(1-methylethyl)cyclohexyl)carbonyl)alanine ethyl ester. Menthyl esters including those of amino acids such as glycine and alanine are disclosed e.g., in EP 310,299 and in U.S. Pat. Nos. 3,111,127; 3,917,613; 3,991,178; 5,703,123; 5,725,865; 5,843,466; 6,365,215; 6,451,844; and 6,884,903. Ketal derivatives are described, e.g., in U.S. Pat. Nos. 5,266,592; 5,977,166 and 5,451,404. Additional agents that are structurally unrelated to menthol but have been reported to have a similar physiological cooling effect include alpha-keto enamine derivatives described in U.S. Pat. No. 6,592,884 including 3-methyl-2-(1-pyrrolidinyl)-2-cyclopenten-1-one (3-MPC), 5-methyl-2-(1-pyrrolidinyl)-2-cyclopenten-1-one (5-MPC), and 2,5-dimethyl-4-(1-pyrrolidinyl)-3(2H)-furanone (DMPF); icilin (also known as AG-3-5, chemical name 1-[2-hydroxyphenyl]-4-[2-nitrophenyl]-1,2,3,6-tetrahydropyrimidine-2-one) described in Wei et al., J. Pharm. Pharmacol. (1983), 35:110-112. Reviews on the coolant activity of menthol and synthetic coolants include H R. Watson, et al. J. Soc. Cosmet. Chem. (1978), 29, 185-200 and R. Eccles, J. Pharm. Pharmacol., (1994), 46, 618-630.

### IV. Encapsulated Actives

In one embodiment, the skin engaging member of the present invention further comprises at least one encapsulated active. The encapsulated active can be an anti-irritation agent as described above, a thermally resilient skin care active or another skin care composition, such as a cooling agent. In one embodiment, the level of said at least one encapsulated active (including the weight of the capsule and encapsulated active) is from about 0.01% to about 50% by weight of said skin engaging member, alternatively from about 10% to about 45%, alternatively from about 15% to about 35%. The encapsulated actives can contain the same ingredients or different ingredients. The encapsulated actives can also include mixtures of ingredients.

The encapsulated can be a cyclodextrin inclusion complex such as described in U.S. Pat. Nos. 5,653,971, and, 5,713,131 and/or another encapsulation technology. The anti-irritation agents of the present invention can be included as a neat ingredient (as a direct addition into the composition) and/or in an encapsulate. In one embodiment, one or more of the anti-irritation agents can be present in both a neat form and in an encapsulate. In one embodiment, one of the anti-irritation agents can be in a neat form and another can be in a capsule.

In one embodiment, encapsulated active comprises more than one cooling agent, for example L-menthol+Menthyl lactate (Frescolat ML); L-menthol+Menthone Glycerine Acetal (Frescolat MGA); or L-menthol+Coolact 10. In yet another embodiment, the encapsulated active comprises at least one cooling agent and a fragrance, a mineral oil, or a combination thereof. In another embodiment, the cooling agent comprises a mixture of menthol and menthyl lactate, such as described in WO 2007115593 (commercially available as Fresocolat Plus), or the eutectic mixture of menthol and menthyl lactate in a ratio of weight in the range of 1:4 to 4:1, as described in U.S. Pat. No. 6,897,195.

Suitable cooling agents which can be utilized include non-volatile menthol analogs such as menthyl lactate, menthyl ethoxyacetate, menthone glycerinacetal, 3-1menthoxypropane-1,2-diol, ethyl 1-menthyl carbonate, (IS,3S,4R)-p-menth-8-en-3-ol, menthyl pyrrolidone 25 carboxylate, N-substituted-p-menthane-3-carboxamides (as described in U.S. Pat. No. 4,136,163, which is incorporated herein by reference) including, for example, N-ethyl-pmenthane-3-carboxamide, acyclic carboxamides Suitable skin-soothing agents which can be utilized in the cyclodextrin inclusion complex include menthol, camphor, eugenol, eucalyptol, safrol, methyl salicylate, and the aforedescribed menthol analogs. Any suitable cyclodextrin may be utilized to form the inclusion complex including alphacyclodextrin, beta-cyclodextrin, gamma-cyclodextrin and modified cyclodextrins such as hydroxypropyl-beta-cyclodextrin, methyl-beta-cyclodextrin., and acetyl-betacyclodextrin. The preferred cyclodextrins are betacyclodextrin and gamma-cyclodextrin.

When the matrix material comprises a cyclodextrin inclusion complex, the matrix material may also advantageously comprise up to 65 about 10%, preferably about 2 to 7%, by weight of a displacing agent which displaces the skin-soothing agent from the inclusion complex upon contact with water, thereby enhancing the release of the skin-soothing agent from the skin engaging member material during use. The displacing agent is a material which is capable of forming a more stable complex with the cyclodextrin than the complex formed with the skin soothing agent and, thus, displaces the skin-soothing agent from the complex when the skin engaging member is contacted with water. Suitable displacing agents include surfactants, benzoic acids, and certain amines (e.g. urea). Further details with respect to the aforementioned cooling agents, cyclodextrin inclusion complexes and displacing agents may be found in U.S. Pat. Nos. 5,653,971, and, 5,713,131.

Nonlimiting examples of encapsulation technology other than cyclodextrin complexes include the nano and micro particles described in U.S. Pat. No. 7,115,282. The nano-particles of the present invention are hydrophobic in nature. In one embodiment, the nano-particles have an average diameter in the range from about 0.01 micron to about 10 microns, or from about 0.05 microns to about 5 microns, or from about 0.1 microns to about 2 microns. This linear dimension for any individual particle represents the length of the longest straight line joining two points on the surface of the particle. In one embodiment, a portion of the nano-particles are encapsulated into one or more water-sensitive micro-particles. In one embodiment, the majority of the nano-particles present in the skin engaging member are encapsulated into said water-sensitive micro-particles. The micro-particles have an average particle size of from about 2.0 microns to about 100 microns, or from 20 microns to about 100 microns.

In one embodiment the level of active or actives in the encapsulated active ranges from about 20 to about 90%, preferably from about 30% to about 75% by weight of the nano-particles. In one embodiment the level of the active or actives in the encapsulated active ranges from about 10% to about 60%, or from about 30% to about 50% by weight of the micro-particles. Lower levels of the encapsulated active can also be used, such as low as 10%, or as low as 5%, or as low as 1%.

### V. Matrix Material

The skin engaging further comprises a matrix material within which said at least one anti-irritation agent is present. As explained above, the agent can be included in a neat form, or can be encapsulated. The matrix material can be in the form of a solid polymeric matrix or an emollient.

g. Solid Polymeric Matrix

In one embodiment, the skin engaging member comprises a solid polymeric matrix comprising a water-soluble polymer material having a melting point of from about 150° C. to about 250° C. and optionally a water-insoluble polymer material In one embodiment, the matrix comprises a water soluble polymer comprising at least one of a polyethylene oxide, polyvinyl pyrrolidone, polyacrylamide, polyhydroxymethacrylate, polyvinyl imidazoline, polyethylene glycol, polyvinyl alcohol, polyhydroxyethymethacrylate, silicone polymers, and a mixtures thereof. In one embodiment, said water soluble polymer is selected from the group consisting of polyethylene oxide, polyethylene glycol, and a mixture thereof.

In one embodiment, the skin engaging member comprises any other ingredients commonly found in commercially available skin engaging members, such as those used on razor cartridges by Gillette, Schick or BIC. Non-limiting examples of such skin engaging members include those disclosed in U.S. Pat. No. 6,301,785, 6,442,839, 6,298,558, 6,302,785, and U.S Patent Pubs. 2008/060201, and 2009/0223057. In one embodiment, the skin engaging member further comprises a skin engaging member ingredient selected from the group consisting of polyethylene oxide, polyvinyl pyrrolidone, polyacrylamide, hydroxypropyl cellulose, polyvinyl imidazoline, polyethylene glycol, poly vinyl alcohol, polyhydroxyethylmethacrylate, silicone copolymers, sucrose stearate, vitamin E, soaps, surfactants, panthenol, aloe, plasticizers, such as polyethylene glycol; beard softeners; additional lubricants, such as silicone oil, Teflon® polytetrafluoroethylene powders (manufactured by DuPont), and waxes; essential oils such as menthol, camphor, eugenol, eucalyptol, safrol and methyl salicylate; tackifiers such as Hercules Regalrez 1094 and 1126; non-volatile cooling agents, inclusion complexes of skin-soothing agents with cyclodextrins; fragrances; antipruritic/counterirritant materials; antimicrobial/keratolytic materials such as Resorcinol; anti-inflammatory agents such as Candilla wax and glycyrrhetinic acid; astringents such as zinc sulfate; surfactants such as pluronic and iconol materials; compatibilizers such as styrene-b-EO copolymers; mineral oil, polycaprolactone (PCL), and combinations thereof.

The water-soluble polymer will preferably comprise at least 50%, more preferably at least 60%, by weight of the skin engaging member, up to about 99%, or up to about 90% of the matrix. The more preferred water soluble polymers are the polyethylene oxides generally known as POLYOX (available from Dow or ALKOX (available from Meisei Chemical Works, Kyoto, Japan). These polyethylene oxides will preferably have mol.wt.s of about 100,000 to 6 million, most preferably about 300,000 to 5 million. The most preferred polyethylene oxide comprises a blend of about 40 to 80% of polyethylene oxide having an average mol.wt. of about 5 million (e.g. POLYOX COAGULANT) and about 60 to 20% of polyethylene oxide having an average mol.wt. of about 300,000 (e.g. POLYOX WSR-N-750). The polyethylene oxide blend may also advantageously contain up to about 10% by weight of a low mol.wt. (i.e. MW<10,000) polyethylene glycol such as PEG-100.

In one embodiment, the matrix further comprises from about 0.5% to about 50%, preferably from about 1% to about 20%, polycaprolactone (preferably mol.wt. of 30,000 to 60,000 daltons). See U.S. Pat. No. 6,302,785.

In another embodiment, the skin engaging member may contain other conventional skin engaging member ingredients, such as low mol.wt. water-soluble release enhancing agents such as polyethylene glycol (MW<10,000, e.g., 1-10% by weight PEG-100), water-swellable release enhancing agents such as cross-linked polyacrylics (e.g., 2-7% by weight), colorants, antioxidants, preservatives, vitamin E, aloe, cooling agents, essential oils, beard softeners, astringents, medicinal agents, etc. Portions that contain a colorant can be designed to release the colorant (e.g., by leaching or abrasion), and thereby cause the strip to change color during shaving, preferably in response to wear of the colored portion, so as to provide an indication to the user that the skin engaging member and/or the razor cartridge has reached the end of its effective life or the end of its optimum performance. A portion may contain, for example, between about 0.1% and about 5.0% (preferably between about 0.5% and 3%) colorant by weight.

The matrix can further comprise a water-insoluble polymer in which the water-soluble polymer is dispersed. Preferably, at a level of from about 0% to about 50%, more preferably about 5% to about 40%, and most preferably about 15% to about 35% by weight of the skin engaging member of a water-insoluble polymer. Suitable water-insoluble polymers which can be used include polyethylene (PE), polypropylene, polystyrene (PS), butadiene-styrene copolymer (e.g. medium and high impact polystyrene), polyacetal, acrylonitrile-butadiene-styrene copolymer, ethylene vinyl acetate copolymer, polyurethane, and blends thereof such as polypropylene/polystyrene blend or polystyrene/impact polystyrene blend.

One preferred water-insoluble polymer is polystyrene, preferably a general purpose polystyrene or a high impact polystyrene such as Styrenics 5410 from Ineos (i.e. polystyrene-butadiene), such as BASF 495F KG21. The strip or any portion should contain a sufficient quantity of water-insoluble polymer to provide adequate mechanical strength, both during production and use.

### VI. Hair Removal Head

The hair removal device generally comprises a hair removal head and a handle or grip portion, upon which the hair removal head is mounted. The hair removal device can be a manual or power driven and can be used for wet and/or dry application. The hair removal head can include a wide scraping surface such as where the hair removal device is used with a depilatory, or a razor cartridge where the device is a shaving razor. The hair removal head may be replaceable or pivotally connected to a cartridge connecting structure. In an aspect, the cartridge connecting structure includes at least one arm to releasably engage the hair removal head.

The hair removal head comprises one or more elongated edges positioned between said first and said second end, said one or more elongated edges comprising a tip extending towards said first end. Where the hair removal head is a razor cartridge the one or more elongated edges can include blades. For example, U.S. Pat. No. 7,168,173 generally describes a Fusion® razor that is commercially available from The Gillette Company which includes a razor cartridge with multiple blades. Additionally, the razor cartridge may include a guard as well as a skin engaging member. A variety of razor cartridges can be used in accordance with the present invention. Non-limiting examples of suitable razor cartridges, with and without fins, guards, and/or shave aids, include those marketed by The Gillette Company under the Fusion®, Venus® product lines as well as those disclosed in U.S. Pat. Nos. 7,197,825, 6,449,849, 6,442,839, 6,301,785, 6,298,558; 6,161,288, and U.S. Patent Publ. No. 2008/060201. Those of skill in the art will understand that the present skin engaging member can be used with any currently marketed system or disposable razor, including those having 1, 2, 3, 4 or 5 blades. Another example of a hair removal device is a scraping edge for use with a hair removal composition, i.e. a depilatory.

In one embodiment, said at least one skin engaging member is located on the portion of the cartridge that contacts skin during the hair removal process, forward and/or aft of the blades. A feature “forward” of the one or more elongated edges, for example, is positioned so that the surface to be treated with by the hair removal device encounters the feature before it encounters the elongated edges. A feature “aft” of the elongated edge is positioned so that the surface to be treated by the hair removal device encounters the feature after it encounters the elongated edges. Where more than one skin engaging members is provided on the hair removal device, they can be the same or different. By different, meaning having a different carrier, a different skin engaging member, or wherein both sheath and composition are different.

In one embodiment, the cartridge comprises a guard comprising at least one elongated flexible protrusions to engage a user's skin. In one embodiment, at least one flexible protrusions comprises flexible fins generally parallel to said one or more elongated edges. In anther embodiment, said at least one flexible protrusions comprises flexible fins comprises at least one portion which is not generally parallel to said one or more elongated edges. Non-limiting examples of suitable guards include those used in current razor blades and include those disclosed in U.S. Pat. Nos. 7,607,230 and 7,024,776; (disclosing elastomeric/flexible fin bars); U.S. Patent Publ. Nos. 2008/0034590 (disclosing curved guard fins); 2009/0049695A1 (disclosing an elastomeric guard having guard forming at least one passage extending between an upper surface and a lower surface). In one embodiment, said skin engaging member is positioned on the cartridge aft of the guard and forward of said elongated edge. In another embodiment, the skin engaging member is positioned on the cartridge forward of the guard. This embodiment can be particularly useful to deliver the skin engaging member prior to contact with the guard.

### VII. Method of Making

Skin engaging member of the present invention is made by extrusion or another high temperature processing, such as injection molding, compacting, ultrasonic or radio frequency sintering, and slot coating. In one embodiment, the skin engaging member comprising the anti-irritation agent of the present invention can further be coated or layered with another shaving aid. In one embodiment, all of the components of the strip, including the anti-irritation agent can be blended prior to molding or extrusion. It can be preferred that the components are free flowing powders, however, liquid skin actives may be adsorbed onto one or more of the other components in the strip that are in powder form

The blended components may be extruded through a Haake System 90, ¾ inch diameter extruder with a barrel pressure of about 1000-2000 psi, a rotor speed of about 10 to 50 rpm, and a temperature of about 150°-185° C. and a die temperature of about 170°-185° C. Alternatively, a 1¼ inch single screw extruder may be employed with a processing temperature of 175°-200° C., preferably 185°-190° C., a screw speed of 20 to 50 rpm, preferably 25 to 35 rpm, and an extrusion pressure of 1800 to 5000 psi, preferably 2000 to 3500 psi. The extruded strip is air cooled to about 25° C. To injection mold the strips it is preferred to first extrude the powder blend into pellets. This can be done on a 1¼ or 1½ inch single screw extruder at a temperature of 120°-180° C., preferably 140°-150° C., with a screw speed of 20 to 100 rpm, preferably 45 to 70 rpm. The pellets are then molded in either a single material molding or multi-material molding machine, which may be single cavity or multi-cavity, optionally equipped with a hot-runner system. The process temperature can be from 165° to 250° C., preferably from 180° to 225° C. The injection pressure should be sufficient to fill the part completely without flashing. Depending on the cavity size, configuration and quantity, the injection pressure can range from 300 to 2500 psi. The cycle time is dependent on the same parameters and can range from 3 to 30 seconds, with the optimum generally being about 6 to 15 seconds. In one embodiment, one or more feeds can be preheated or they can be fed in at ambient temperature.

### VIII. Details on Figures

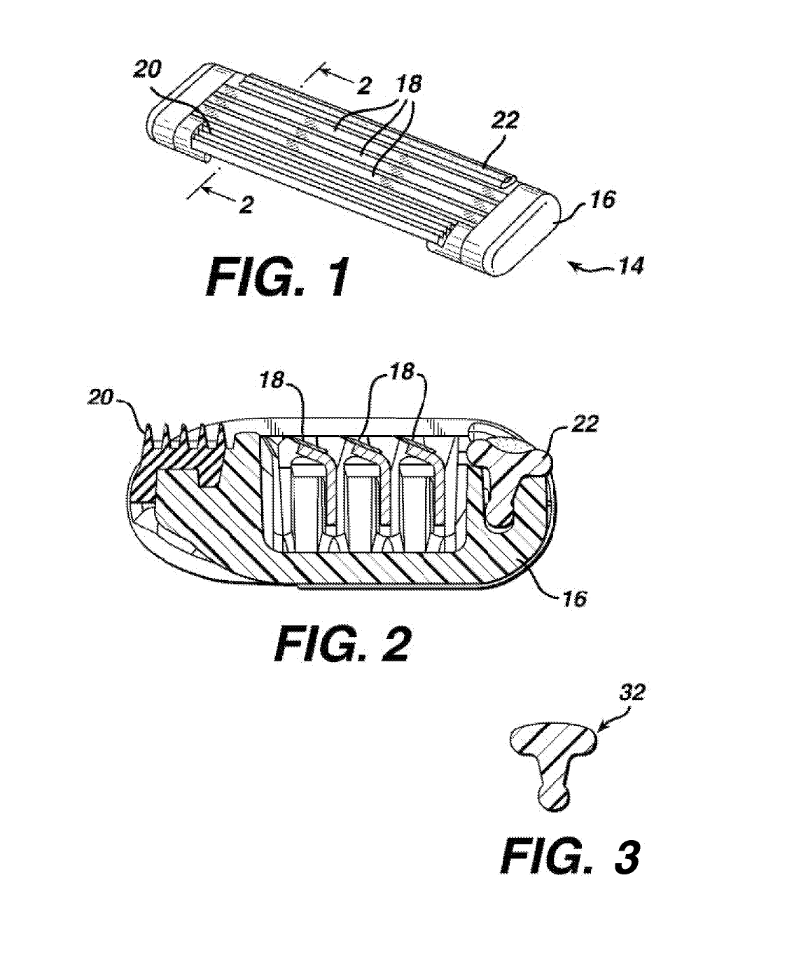

Referring to FIGS. 1 and 2, the razor cartridge 14 includes housing 16, which carries three blades 18, a finned elastomeric guard 20, and a skin engaging member 22 located on a skin-engaging portion (in this case the cap) of the cartridge. Skin engaging member 22 is shown having two layers, the first layer can be the matrix and encapsulated active of the present invention, and the second layer can be a conventional shave aid, or vice versa. The skin engaging member is preferably locked in (via adhesive, a fitment, or melt bonding) an opening or on a plate or other flat surface in the rear of the cartridge. Skin engaging member 32, shown in FIG. 3, is similar to skin engaging member 22, except that skin engaging member 32 has a homogeneous composition throughout and a uniform, slightly curved to flat upper surface.

### IX. Examples

The following examples are made in accordance with the present invention. Each of the examples can include varying levels of anti-irritation agent, such as zinc pyrithione, such as from 0.5% to about 3%. Zinc Oxide can also be included in such examples at levels from 1% to about 5%. Various other levels disclosed above can also be used.

### Example 1

| Material | Wt % |
|---|---|
| Dow Polyox N-750 | 23.94 |
| Dow Polyox Coagulant | 35.91 |
| Carbowax 4600 PEG | 4.75 |
| White Concentrate | 1.14 |
| Irganox Antioxidant | 0.25 |
| HIPS 371G with acrowax | 29.01 |
| Paltenex ® (Peptide) | 5.00 |

### Example 2

| Material | Wt % |
|---|---|
| Dow Polyox N-750 | 22.89 |
| Dow Polyox Coagulant | 34.34 |
| Carbowax 4600 PEG | 4.54 |
| White Concentrate | 1.09 |
| Irganox Antioxidant | 0.23 |
| HIPS 371G with acrowax | 27.73 |
| Maxi-lip ® (Peptide) | 9.18 |

### Example 3

| Material | Wt % |
|---|---|
| Dow Polyox N-750 | 23.94 |
| Dow Polyox Coagulant | 35.92 |
| Carbowax 4600 PEG | 4.75 |
| White Concentrate | 1.14 |
| Irganox Antioxidant | 0.24 |
| HIPS 371G with acrowax | 29.01 |
| Net-DG ® (glycyrrhizinate salt) | 5.00 |

### Example 4

| Material | Wt % |
|---|---|
| Dow Polyox N-750 | 23.94 |
| Dow Polyox Coagulant | 35.92 |
| Carbowax 4600 PEG | 4.75 |
| White Concentrate | 1.14 |
| Irganox Antioxidant | 0.24 |
| HIPS 371G with acrowax | 29.01 |
| Olivem 460 ® (Surfactant | 5.00 |
| derived from a Triglyceride) | |

### Example 5

| Material | % |
|---|---|
| Dow Polyox N-750 | 23.2 |
| Dow Polyox Coagulant | 34.81 |
| Carbowax 4600 PEG | 5.00 |
| White Concentrate | 1.20 |
| Irganox Antioxidant | 0.25 |
| HIPS 371G with acrowax | 30.54 |
| Ceramidone (Ester of PCA) | 5.00 |

### Measuring Molecular Survival Via GC-MS

Peptide containing skin engaging members such as Examples for 2 can be tested for molecular survival. Further, a sample skin engaging member comprising Pal-GHK can be tested. The peptide is extracted from the skin engaging member by sonicating in 50/50/1 methanol/water/formic acid for 120 minutes. A control skin engaging member (without the peptide but with all other ingredients at the same proportion) is carried through the sample procedure to evaluate matrix interference. The peptide (i.e. Pal-GHK) is subjected to reverse phase liquid chromatography on an appropriate column such as a Waters XBridge Phenyl column (2.1×50 mm, 2.5 μm particles). Detection and quantitation is by mass spectrometry operating under multiple reaction monitoring (MRM) MS/MS conditions in the positive-ion mode.

Net-DG can be extracted from a skin engaging member of Example 3 by sonicating in methanol for 120 minutes. In addition, a control skin engaging member is carried through the same procedure to evaluate matrix interference. Net-DG is subjected to reverse phase liquid chromatography on an appropriate column such as a Waters XBridge Shield RP18 column (2.1×50 mm, 2.5 μm particles). Detection and quantitation is by mass spectrometry operating under multiple reaction monitoring (MRM) MS/MS conditions in the positive-ion mode.

Components of OliveM can be extracted from a skin engaging member of Example 4 by sonicating in methanol for 120 minutes. In addition, a control skin engaging member without Olivem is carried through the same procedure to evaluate matrix interference. OliveM components are subjected to reverse phase liquid chromatography on an appropriate column such as a Ace 5 C8 column (2.1×50 mm, 5 μm particles). Detection and quantitation is by mass spectrometry operating under multiple reaction monitoring (MRM) MS/MS conditions in the positive-ion mode.

Ceramidone can be extracted from a skin engaging member of Example 5 by sonicating in methanol, such as for 120 minutes. A control skin engaging member (without Ceramidone) was carried through the sample procedure to evaluate matrix interference. Ceramidone is subjected to reverse phase liquid chromatography on an appropriate column such as a Waters XBridge Shield RP18 column (2.1×50 mm, 2.5 μm particles). Detection and quantitation is by mass spectrometry operating under multiple reaction monitoring (MRM) MS/MS conditions in the positive-ion mode.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the DETAILED DESCRIPTION OF THE INVENTION are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern. Except as otherwise noted, the articles “a,” “an,” and “the” mean “one or more.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin engaging member for use on a hair removal device, said skin engaging member comprising:
a. a solid polymeric matrix comprising a water-soluble polymer material and a water-insoluble polymer material, said solid polymeric matrix having a melting point of from about 120° C. to about 200° C.; and
b. from about 0.01% to 5% of an anti-irritation agent comprising a pyrithione, a polyvalent metal salt of pyrithione, and a mixture thereof.

2. The skin engaging member of claim 1, wherein said level of anti irritation agent is from about 0.25% to about 2.5%.

3. The skin engaging member of claim 1, wherein said anti-irritation agent comprises a zinc pyrithione.

4. The skin engaging member of claim 3, further comprising from 0.001% to 10% of a zinc salt.

5. The skin engaging member of claim 3, wherein the zinc pyrithione is in platelet particle form having an average size of up to about 20 μm.

6. The skin engaging member of claim 3, wherein said zinc pyrithione is in the form of a platelet having a median particle diameter of about 0.5 microns to about 10, a mean particle diameter of about 0.5 to about 10 microns, and a thickness of about 0.6 to about 15 microns.

7. The skin engaging member of claim 6, wherein the mean particle diameter of the platelet ZPT is about 2 microns to about 4 microns.

8. The skin engaging member of claim 6, wherein the median particle diameter of the platelet ZPT is about 1 micron to about 5 microns.

9. The skin engaging member of claim 1, further comprising at least one thermally resilient skin care active comprising at least one of: a glycyrrhizinate salt; a surfactant derived from a triglyceride; a ceramide, an esters of PCA; a peptide; and a mixture thereof.

10. The skin engaging member of claim 1, wherein said level of water soluble polymer is at a level of from about 50% to about 100% by weight of said solid polymeric matrix, and wherein said water soluble polymer of the matrix comprises at least one of a polyethylene oxide, polyvinyl pyrrolidone, polyacrylamide, polyhydroxymethacrylate, polyvinyl imidazoline, polyethylene glycol, polyvinyl alcohol, polyhydroxyethymethacrylate, silicone polymers, and mixtures thereof.

11. The skin engaging member of claim 1, wherein said matrix further comprises a water insoluble polymer at a level of at least about 35% by weight of said solid polymeric matrix, said water insoluble polymer comprising at least one of: polyethylene, polypropylene, polystyrene, high impact polystyrene, butadiene styrene copolymer, polyacetal, acrylonitrile-butadiene styrene copolymer, ethylene vinyl acetate copolymer, and mixtures thereof.

12. The skin engaging member of claim 1, wherein said matrix material comprises an emollient.

13. A method of making a skin engaging member comprising the steps of:
a. providing a polymeric matrix and an anti-irritation agent comprising a zinc pyrithione to form a feed; and
b. extruding said feed to form a skin engaging member.

14. The method of claim 13, further comprising a step of heating said feed to a temperature of from about 120° C. to about 200° C. prior to extruding.

15. The method of claim 14, wherein the step of heating occurs during the extruding step.

16. A hair removal device comprising:
a. a cartridge;
b. one or more elongated edges positioned on said cartridge; and the skin engaging member, said skin engaging member comprising:
i. a solid polymeric matrix comprising a water-soluble polymer material and a water-insoluble polymer material, said solid polymeric matrix having a melting point of from about 120° C. to about 200° C.; and
ii. from about 0.01% to 5% of an anti-irritation agent comprising a pyrithione, a polyvalent metal salt of pyrithione, and a mixture thereof.

17. The hair removal device of claim 16, further comprising a handle, upon which said cartridge is mounted.

18. The skin engaging member of claim 1, wherein the anti-irritation agent is a solid particle in platelet form having an average size of up to about 20 μm.

19. The skin engaging member of claim 18, wherein said solid particle is in the form of a platelet having a median particle diameter of about 0.5 microns to about 10, a mean particle diameter of about 0.5 to about 10 microns, and a thickness of about 0.6 to about 15 microns.

20. The skin engaging member of claim 19, wherein the mean particle diameter of the platelet is about 2 microns to about 4 microns.
